(19) United States
(12) Patent Application Publication
(10) Pub. No.: US 2025/0177045 A1
(43) Pub. Date: Jun. 5, 2025

(54) METHOD FOR DETERMINING INTERVERTEBRAL IMPLANT DEVICE PARAMETERS OF AN INTERVERTEBRAL IMPLANT DEVICE FOR SPINAL FUSION

(71) Applicant: MEDACTA INTERNATIONAL SA, CASTEL SAN PIETRO (CH)

(72) Inventors: Francesco SICCARDI, CASTEL SAN PIETRO (CH); Meinrad FIECHTER, CASTEL SAN PIETRO (CH); Athos AMBROSINI, CASTEL SAN PIETRO (CH)

(21) Appl. No.: 18/844,796

(22) PCT Filed: Mar. 6, 2023
(86) PCT No.: PCT/IB2023/052061

§ 371 Date: Sep. 6, 2024

(30) Foreign Application Priority Data
Mar. 8, 2022 (IT) 102022000004367

(51) Int. Cl. A61B 34/10; G06T 19/00; G16H 50/50

(52) U.S. Cl. CPC A61B 34/10; G06T 19/00; G16H 50/50; A61B 2034/104; A61B 2034/105; G06T 2210/41

(57) ABSTRACT

A method for determining intervertebral implant device parameters includes a) acquiring a tridimensional image of a spine of a patient; b) calculating a tridimensional model of the spine; c) acquiring at least one bidimensional image of the spine; d) performing a first sagittal balance of the tridimensional model to calculate spino-pelvic parameters; e) determining intervertebral device parameters corresponding to intervertebral devices to be used; f) applying the intervertebral device parameters to the tridimensional model of the spin, thus obtaining a corrected tridimensional model of the spine; g) comparing the corrected tridimensional model of the spine with the bidimensional image and performing a second sagittal balance obtaining corrected spino-pelvic parameters; h) comparing the corrected parameters with predetermined values to determine if the desired balanced spine has been obtained; and i) in negative case, repeating steps e)-h).

The present invention relates to a method for determining intervertebral implant device parameters of an intervertebral implant device for spinal fusion.

Spinal fusion is a surgery treatment to permanently connect two or more vertebrae of a spine, thus eliminating motion between them for stabilizing a vertebral segment of a patient.

Spinal fusion surgery is a medical treatment wherein interbody cages can be placed into the spine of a patient. The cages serve as a space holder between affected vertebrae and allows bone to grow through them, eventually becoming a part of the spine.

Interbody cages are placed between the bodies of two adjacent vertebrae, after removing the intervertebral disc that typically occupies this space. Spinal cages can be made of metal, polymer, ceramic, or a fusion of different materials.

The cages have a hollow center, which is filled with a bone-growth promoting material or the own bone of the patient taken from, for example, the hip during the same surgery as the fusion.

Interbody cages aim to improve the stability and balance of the treated spinal segment, while also relieving pain and restoring function.

Cage selection for intervertebral body fusion is general done intra-operative based on simple mechanical trials. These trails allow a basic geometrical analysis about the shape and fit, but they do not allow considering many important factors like sagittal balance, facet interference, foraminal height and implant endplate contact.

Nowadays, technology does not provide any tool to plan, before a surgical operation, the characteristics of cages or other implant devices to use in order to obtain a desired correction of a damaged spine.

There is therefore the need to have a possibility to perform a planning of a spine surgical operation, by determining physical and constructive features of devices to implant in order to achieve a desired correction of a defeat of the spine, thus overcoming the problems of the prior art.

These and other objects are fully achieved by virtue of a method for determining intervertebral implant device parameters of an intervertebral implant device for spinal fusion, having the characteristics defined in independent claim 1, and by a system for determining intervertebral implant device parameters of an intervertebral implant device for spinal fusion, having the characteristics defined in claim 10.

Preferred embodiments of the invention are specified in the dependent claims, whose subject-matter is to be understood as forming integral or integrating part of the present description.

Further characteristic and advantages of the present invention will become apparent from the following description, provided merely by way of non-limiting example, with reference to the attached drawings, in which:

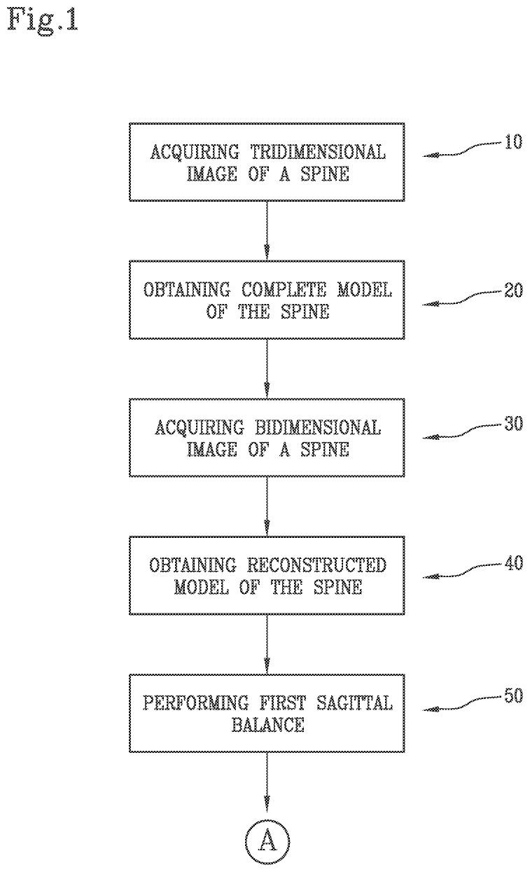

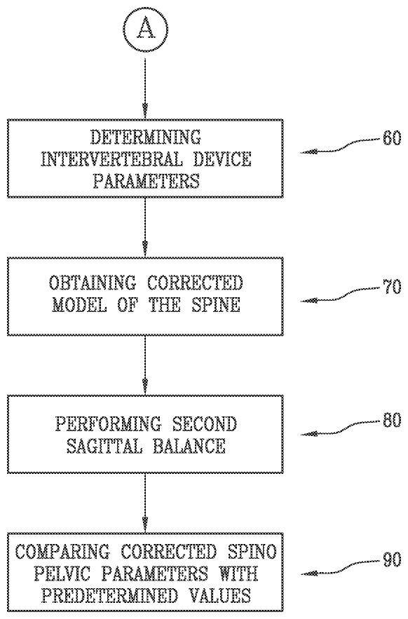

FIG. 1 shows a block diagram of the step of a method for determining intervertebral implant device parameters of an intervertebral implant device for spinal fusion according to the present invention; and

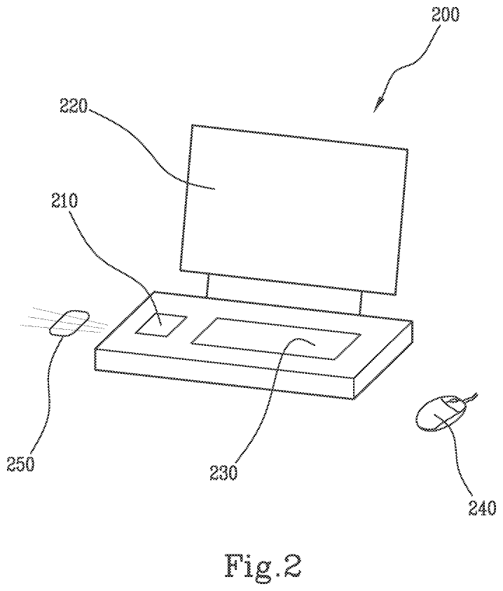

FIG. 2 shows a system for determining intervertebral implant device parameters of an intervertebral implant device for spinal fusion according to the present invention.

Briefly, in the method for determining intervertebral implant device parameters of an intervertebral implant device for spinal fusion according to the present invention, patient specific analysis of the impact of a movement of tridimensional reconstructed vertebras and the interaction between the patient anatomy with an implant device are performed, based on a tridimensional scan of the patient spine, such as CT or MRI scans.

An amount of correction is defined, based on a sagittal balance analysis, taking into account implant devices such as cages with different heights and lordotic/kyphotic angles, inserted inside the disc space and affecting the geometrical relationship of two vertebras, e.g., distance, translation, rotation and angle (lordosis/kyphosis).

An optimal selection of the device to implant can therefore be obtained, considering its design, length, height, width and lordosis, taking into account different factors such as the sagittal balance, the foraminal height/indirect decompression, the facet interference caused by intervertebral movement, the implant/vertebral endplate contact, etc.

In case of performing of a CT scan, the bone density in the area of the implant endplate contact, using Hounsfield Units, is also available, provided within the CT scan information.

In addition, planned information can be used in an intra-operative monitor to check if the execution of the surgical operation is according to the plan or to identify any gap between a planned and an actual foraminal height.

FIG. 1 shows a block diagram of the step of a method for determining intervertebral implant device parameters of an intervertebral implant device for spinal fusion according to the present invention.

In a first step 10, a tridimensional scan image of the spine of a patient is acquired, for example a MRI or CT image.

In a further step 20, a tridimensional model of at least one vertebra is created, in a manner per se known, starting from the scan image of the spine of step 10. In an embodiment of the invention, a tridimensional virtual complete model of the spine is obtained, in particular as the juxtaposition of a plurality of individual singular vertebrae. Advantageously, tridimensional images of a lower extremity of the spine (sacrum and/or pelvis) are added to the complete model of the spine.

In a third step 30, at least one bidimensional x-ray image of the spine of the patient is acquired, preferably a lateral and/or an anterior-posterior (sagittal) x-ray image. The bidimensional image includes a plurality of vertebrae.

In case only limited tridimensional scan information is available, in a next step 40, a matching of the bidimensional and tridimensional images is performed. Tridimensional reconstructed vertebra/sacrum/pelvic images are selected from a predefined database of patient specific tridimensional vertebrae, wherein the selected images have a predetermined match with the vertebrae images of the bidimensional x-ray image of step 30, and then they are combined, to obtain a reconstructed model of the spine combining the bidimensional x-ray with tridimensional information for the levels the information is available

In step 40, the tridimensional images of the reconstructed vertebrae of the database are matched, for example by using a DDR (Digital Reconstructed Radiograph) algorithm, to the bidimensional images of the vertebrae of the x-ray acquisition, in order to obtain a tridimensional virtual reconstructed model of the spine of the patient.

At this point, the positional change among vertebras and the potential interaction between an implant and the patient anatomy are analysed.

In a step 50, either the x-ray image or the tridimensional model of the spine (complete or reconstructed) is used to perform a first sagittal balance analysis to calculate spino pelvic parameters of the patient such as pelvic incident, sacral slope, pelvic tilt, lumbar lordosis, thoracic kyphosis, sagittal vertical axis.

Advantageously, additional information like a historical patient disc height analysis or an analysis of the disc height on the healthy level above below the index level are considered, to better predict a possible amount of distraction and, as a consequence, the implant height and lordosis on the index level.

The values of the spino-pelvic parameters are used, in a further step 60, to analyse the relationship and movement between different vertebrae of the tridimensional model of the spine, for example, the lordosis or kyphosis correction which has to be applied on the different levels of the spine to get a final balanced spine. This allows determining, in a manner per se known, intervertebral device parameters such as an optimal geometry (design, length, height, width, lordosis, kyphosis), a bone mineral density, an end plate contact and a facet interference of an intervertebral device such as a cage to be used in surgery to get the desired balanced spine.

These intervertebral device parameters have to be adequate to components commercially available to the market because the surgeon will choose, among possible devices to use for the spinal fusion, the one which best matches the intervertebral device parameters above calculated.

In a further step 70, the intervertebral device parameters are applied, in a manner per sè known, to the tridimensional model of the spine (complete or reconstructed), thus obtaining a corrected tridimensional model of the spine.

In a step 80, the corrected tridimensional model of the spine is aligned with the bidimensional x-ray image to ensure the correction defined is present in the X-Ray and the 3D model, in order to update sagittal balance analysis performed in step 50, if needed. A second sagittal balance analysis is performed, thus obtaining corrected spino pelvic parameters (corrected with respect to the spino pelvic parameters calculated at step 50 thanks to the intervertebral device of the tridimensional model of the spine). In case the segmental lordosis defined in step 50 does not change in the tridimensional model, the spino pelvic parameters remain the same as previously calculated.

In a step 90, the corrected spino pelvic parameters are compared with predetermined range values reported in literature, in order to determine if the desired balanced spine has been obtained.

In positive case, i.e. the corrected spino pelvic parameters are within such range values, the intervertebral device parameters above determined can be used to design a corresponding intervertebral device to apply to the spine of the patient, during a subsequent surgical operation.

In negative case, steps 60-90 are repeated until correct intervertebral device parameters are obtained.

The process according to the invention is performed by a system of the type illustrated in FIG. 2 comprising a workstation 200 of known type having a processing subsystem 210, a display device 220, a keyboard 230, a pointing device (mouse) 240, and a device for connecting to a local area network (network bus) 250. Alternatively, the processing system may be of a distributed type (not illustrated) having a processing subsystem and local or remote peripheral input/output devices.

The workstation 200 or distributed system is arranged to process processing groups or modules and computational programs that are stored on a disk or are accessible over a network and that are suitable for displaying the described process to display results on the device 220. The described solutions are considered to be well known to one skilled in the art and will therefore not be described herein as they are not relevant to the implementation purposes and understanding of the invention.

Clearly, the principle of the invention remaining the same, the embodiments and the details of production can be varied considerably from what has been described and illustrated purely by way of non-limiting example, without departing from the scope of protection of the present as defined in the attached claims.

1. A method for determining intervertebral implant device parameters of an intervertebral implant device for spinal fusion comprising:
a) acquiring a tridimensional image of a spine of a patient;
b) calculating a tridimensional model of the spine, based on such tridimensional image of the spine;
c) acquiring at least one bidimensional image of the spine;
d) performing a first sagittal balance of the tridimensional model of the spine to calculate spino-pelvic parameters;
e) determining, based on the spino-pelvic parameters, intervertebral device parameters corresponding to intervertebral devices to be used to get a desired balanced spine for the patient;
f) applying the intervertebral device parameters to the tridimensional model of the spine, thus obtaining a corrected tridimensional model of the spine;
g) comparing the corrected tridimensional model of the spine with the bidimensional image and performing a second sagittal balance, thus obtaining corrected spino-pelvic parameters;
h) comparing the corrected spino-pelvic parameters with predetermined range values in order to determine if the desired balanced spine has been obtained; and
i) in a negative case, repeating steps e)-h).

2. The method for determining intervertebral implant device parameters according to claim 1, wherein the tridimensional image of the spine comprises a MRI or CT image.

3. The method for determining intervertebral implant device parameters according to claim 1, wherein the at least one bidimensional image comprises a lateral and/or an anterior-posterior x-ray image.

4. The method for determining intervertebral implant device parameters according to claim 1, wherein calculating a tridimensional model of the spine comprises matching the bidimensional and tridimensional images.

5. The method for determining intervertebral implant device parameters according to claim 4, wherein matching the bidimensional and tridimensional images comprises selecting tridimensional reconstructed images of vertebrae from a predefined database, said selected images having a predetermined match with vertebrae images of the bidimensional image, and then combining the reconstructed images of vertebrae and the bidimensional image to obtain a reconstructed tridimensional model of the spine.

6. The method for determining intervertebral implant device parameters according to claim 5, wherein the step of combining the reconstructed images of vertebrae and the bidimensional image comprises applying a DDR (Digital Reconstructed Radiograph) algorithm.

7. The method for determining intervertebral implant device parameters according to claim 1, wherein the spino-pelvic parameters comprise pelvic incident, sacral slope, pelvic tilt, lumbar lordosis, thoracic kyphosis, sagittal vertical axis.

8. The method for determining intervertebral implant device parameters according to claim 1, wherein the intervertebral device parameters comprise geometry, bone mineral density, end plate contact and facet interference of an intervertebral device.

9. The method for determining intervertebral implant device parameters according to claim 8, wherein the geometry includes design, length, height, width, lordosis and kyphosis of the intervertebral device.

10. A system for determining intervertebral implant device parameters of an intervertebral implant device for spinal fusion comprising a workstation having a processing subsystem arranged to:
a) acquire a tridimensional image of a spine of a patient;
b) calculate a tridimensional model of the spine, based on such tridimensional image of the spine;
c) acquire at least one bidimensional image of the spine;
d) perform a first sagittal balance of the tridimensional model of the spine to calculate spino-pelvic parameters;
e) determine, based on the spino-pelvic parameters, intervertebral device parameters corresponding to intervertebral devices to be used to get a desired balanced spine for the patient;
f) apply the intervertebral device parameters to the tridimensional model of the spine, thus obtaining a corrected tridimensional model of the spine;
g) compare the corrected tridimensional model of the spine with the bidimensional image and perform a second sagittal balance, thus obtaining corrected spino-pelvic parameters;
h) compare the corrected spino-pelvic parameters with predetermined range values in order to determine if the desired balanced spine has been obtained; and
i) in a negative case, repeat steps e)-h).
